(19) United States
(12) Patent Application Publication
(10) Pub. No.: US 2018/0147375 A1
(43) Pub. Date: May 31, 2018

(54) RESUSCITATION AND VENTILATION MONITOR

(71) Applicant: The Regents of the University of California, Oakland, CA (US)

(72) Inventors: Michael Austin Johnson, Davis, CA (US); Justin Koos, Davis, CA (US)

(21) Appl. No.: 15/578,212

(22) PCT Filed: Jun. 2, 2016
(86) PCT No.: PCT/US16/35582

§ 371 Date: Nov. 29, 2017

Related U.S. Application Data
(60) Provisional application No. 62/170,591, filed on Jun. 3, 2015.

(51) Int. Cl. A61M 16/00; A61B 5/087; A61B 5/08; A61B 5/091

(52) U.S. Cl. CPC A61M 16/0051; A61M 16/024; A61B 5/087; A61M 16/0816; A61M 16/0078; A61B 5/091; A61M 16/0084; A61B 5/0816

(57) ABSTRACT

Resuscitation and ventilation monitoring devices are provided. A device includes an inlet in fluid communication with airflows exchanged with lungs of a patient and an airflow meter measuring characteristics of the airflows. A user may provide a controller with patient size information via a measurement selector, enabling the controller to determine acceptable ranges of measured airflow characteristics. If the measured airflow characteristics fall outside an acceptable range, the controller may cause a sensory alarm to alert the user. The device may work within a network of devices and user interfaces. The device may include a variety of sensors including pressure, CO₂, O₂, and temperature sensors. If the measurements of these sensors fall outside an acceptable range, the controller may cause a sensory alarm to alert the user.

### I. CROSS-REFERENCE TO RELATED APPLICATIONS

This application claims the benefit of priority of U.S. Provisional Application No. 62/170,591, filed Jun. 3, 2015, the entire contents of which are incorporated herein by reference.

### II. TECHNICAL FIELD

The present disclosure relates to patient resuscitation and ventilation systems.

### III. BACKGROUND

Airway management is an important aspect of emergency resuscitation. In turn, providing a proper ventilation rate and tidal volume is an important aspect of airway management. Proper ventilation rates and tidal volumes vary along with the overall patient size, gender, and/or age. As such, pediatric airway management may be particularly difficult due to the wide range of heights and weights of pediatric patients.

During emergencies, first responders and clinicians commonly use bag valve masks (“BVM”) or manual resuscitators for airway management. However, many first responders and clinicians inadvertently hyperventilate patients with BVMs or other resuscitation equipment, which may lead to serious complications. Hyperventilation decreases CO₂ in the body of a given patient, which results in alkalosis. Alkalosis impedes the patient's blood hemoglobin to bind to oxygen, which ultimately gives rise to potentially fatal conditions such as cerebral hypoxia and hyperventilation syndrome, which may lead to brain injury and patient mortality. Alkalosis also causes vasoconstriction, which may lead to decreased blood flow to the brain, and has been shown to result in worse outcomes in patients with traumatic brain injuries. Furthermore, inappropriate tidal volumes or the total volume of air that is given with each breath, may lead to barotrauma and the development of Acute Respiratory Distress Syndrome, which leads to increased morbidity and mortality.

Airway management may also be conducted via mechanical ventilation, for example, when the patient is in the hospital. Similarly, mechanical ventilation devices may result in inappropriate respiratory rates and tidal volumes when not appropriately optimized for the patient's size resulting in hypo- or hyper-ventilation as well as barotrauma.

It would therefore be desirable to provide improved systems and methods for airway management.

Specifically, it would be desirable to provide resuscitation and monitoring systems and methods that improve clinical decision support by determining acceptable ranges of measured airflow characteristics.

### IV. SUMMARY

The present disclosure overcomes the drawbacks of previously-known systems by providing systems and methods for improved resuscitation and ventilation monitoring, and enhanced clinical decision support.

One embodiment relates to a resuscitation and ventilation monitoring system. The system includes an inlet in fluid communication with airflows exchanged with lungs of a patient. The system further includes an airflow meter in fluid communication with and collecting airflow measurements from the inlet. The system includes a sensory alarm selectively providing an alert to a user of the system. The system further includes a measurement selector including selectable increments of patient height. The system includes a controller communicatively engaged to each of the airflow meter, the sensory alarm, and the measurement selector. The controller may receive a user input from the measurement selector and determine a corresponding range of acceptable respiratory rates and tidal volumes. The controller may also determine a patient respiratory rate and tidal volumes from the airflow measurements received from the airflow meter. The controller may cause the sensory alarm to provide the alert to the user when at least one of the patient respiratory rate or the tidal volumes is outside the corresponding range of acceptable respiratory rates and tidal volumes.

The system may further include a CO₂ sensor in fluid communication with the inlet, such that the CO₂ sensor may collect CO₂ level measurements from the inlet. In addition, the controller may be communicatively engaged to and receive CO₂ level measurements from the CO₂ sensor. The controller may provide the user with the CO₂ level measurements via a display. The controller may also determine a range of acceptable CO₂ levels and cause the sensory alarm to provide an alert to the user when the CO₂ level measurements are outside the corresponding range of acceptable CO₂ levels.

The system may include an O₂ sensor in fluid communication with the inlet, such that the O₂ sensor may collect O₂ level measurements from the inlet. In addition, the controller may be communicatively engaged to and receive O₂ level measurements from the O₂ sensor. The controller may provide the user with the O₂ level measurements via a display and determine mechanical ventilator settings based on this O₂ level.

The system may include a pressure sensor in fluid communication with the inlet, such that the pressure sensor may collect measurements of the pressure within the system and in the patient's lungs. The pressure sensor may then transmit the pressure information to the controller which may display this output for the user. In addition, the controller may also determine the range of acceptable pressures and cause the sensory alarm to provide an alert to the user when the pressure measurements are outside the corresponding range of acceptable pressure levels.

The sensory alarm may provide a visual alert and/or an audible alert to the user. In addition, the sensory alarm may be coupled to a remote device.

The measurement selector may include a plurality of colored options, wherein each of the plurality colored options correspond to colors and associated measurement increments defined by the Broselow Tape. Each of the colors and associated measurement increments defined by the Broselow Tape may correspond to a respective range of acceptable respiratory rates and tidal volumes. In one embodiment, the measurement selector may include a plurality of switches, wherein each of the plurality switches corresponds to one of the plurality of colored options. In another embodiment, the measurement selector may include a rotatable dial divided into a plurality of segments, wherein each of the plurality of segments corresponds to one of the plurality of colored options. In one embodiment, the measurement selector may include a digital screen configured to display colored digital representations, wherein each colored digital representation corresponds to one of the plurality of colored options. In addition, the measurement selector may also include at least one colored option corresponding to measurement increments for an adult patient, wherein each of the at least one colored option corresponding to measurement increments for an adult patient are associated with a respective range of acceptable respiratory rates and tidal volumes.

The measurement selector may also include selectable increments of at least one of patient weight, patient gender, or patient age.

The controller may be coupled to a remote device. In such an embodiment, the airflow meter may communicate the airflow measurements to the controller via at least one of WiFi, Bluetooth, Wixel-based communication, or cellular communication.

The controller may compare an amount of breath inhaled with an amount of breath exhaled from the airflow measurements received from the airflow meter. In addition, the controller may cause the sensory alarm to provide an alert to the user when the compared amount of breath inhaled and exhaled is outside a predetermined threshold. In one embodiment, the controller may also include a data storage. The data storage may store data comprising at least one of the corresponding range of acceptable respiratory rates, the corresponding range of acceptable tidal volumes, the patient respiratory rate, or the patient tidal volumes. In such an embodiment, the data may be downloaded and analyzed at a later time.

The system may further include a display. The display may provide the user with at least one of a selected increment of patient height, a selected Broselow Tape color, the corresponding range of acceptable respiratory rates and tidal volumes, the patient respiratory rate, the patient CO₂ level, the patient O₂ level, the calculated difference in inhaled and exhaled volume, or the patient total tidal volumes. In addition, the display may provide the user with at least one of the patient respiratory rate or the patient tidal volumes in the form of ventilation waveforms. In such an embodiment, the sensory alarm may provide a visual alert to the user through the display. In addition, the display may be a touchscreen, such that the measurement selector is disposed in the display.

Another embodiment relates to a non-transitory computer-readable medium having instructions that when executed by a processor of a ventilator cause the processor to perform various functions. For example, the executed instructions may cause the processor to receive user input from a user, the user input comprising at least one of patient height, weight, gender, or age, receive airflow measurements indicative of ventilation of a patient from at least one of an airflow meter or one or more sensors, classify ventilation as either on-target or off-target based on whether the ventilation is within a predetermined limit defined by the user input, generate an alert if the ventilation is off-target, and suggest corrective action via a user interface if the alert is generated. In addition, the processor may suggest corrective action that may be implemented by the user to adjust a manual bagging of the patient or ventilator settings of a mechanical ventilator.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### V. BRIEF DESCRIPTION OF THE DRAWINGS

The skilled artisan will understand that the drawings primarily are for illustrative purposes and are not intended to limit the scope of the subject matter described herein. The drawings are not necessarily to scale; in some instances, various aspects of the subject matter disclosed herein may be shown exaggerated or enlarged in the drawings to facilitate an understanding of different features. In the drawings, like reference characters generally refer to like features (e.g., functionally similar and/or structurally similar elements).

FIG. 1 is a schematic block diagram illustrating various features of a resuscitation and ventilation monitoring system, according to an example embodiment.

FIGS. 2A-2D illustrate various arrangements of an airflow meter.

FIG. 3A illustrates an example arrangement of a CO₂ sensor.

FIG. 3B is a schematic diagram illustrating an example arrangement of a band pass filter.

FIGS. 4A-4C illustrate various arrangements of a measurement selector.

FIG. 5A is a schematic block diagram illustrating various features of a controller.

FIG. 5B is a schematic diagram illustrating an example arrangement of a low pass filter.

FIG. 6 is an exploded view of an example embodiment of the resuscitation and ventilation monitoring device of FIG. 1.

FIG. 7 illustrates a flow chart depicting the actions performed by the processor of a ventilation system in accordance with the principles of the present disclosure.

The features and advantages of the inventive concepts disclosed herein will become more apparent from the detailed description set forth below when taken in conjunction with the drawings.

### VI. DETAILED DESCRIPTION

In the following detailed description, reference is made to the accompanying drawings, which form part of the present disclosure. The embodiments described in the drawings and description are intended to be exemplary and not limiting. As used herein, the term “example” means “serving as an example or illustration” and should not necessarily be construed as preferred or advantageous over other embodiments. Other embodiments may be utilized and modifications may be made without departing from the spirit or the scope of the subject matter presented herein. Aspects of the disclosure, as described and illustrated herein, may be arranged, combined, and designed in a variety of different configurations, all of which are explicitly contemplated and form part of this disclosure.

Unless otherwise defined, each technical or scientific term used herein has the same meaning as commonly understood by one of ordinary skill in the art to which this disclosure belongs. In accordance with the claims that follow and the disclosure provided herein, the following terms are defined with the following meanings, unless explicitly stated otherwise.

As used in the specification and claims, the singular form “a”, “an” and “the” include both singular and plural references unless the context clearly dictates otherwise. For example, the term “a sensor” may include, and is contemplated to include, a plurality of sensors. At times, the claims and disclosure may include terms such as “a plurality,” “one or more,” or “at least one;” however, the absence of such terms is not intended to mean, and should not be interpreted to mean, that a plurality is not conceived.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a device or method consisting essentially of the elements as defined herein would not exclude other materials or steps that do not materially affect the basic and novel characteristic(s) of the claimed invention. “Consisting of” shall mean that the devices, systems, and methods include the recited elements and exclude anything more than a trivial or inconsequential element or step. Embodiments defined by each of these transitional terms are within the scope of this disclosure.

“Component,” as used herein, may refer to an individual unit or structure, or it may refer to a portion, feature, or section of a larger structure.

As used herein, “patient” shall mean any individual who receives resuscitation or ventilation treatment.

As used herein, a “user” shall refer to any individual who interacts with, or otherwise uses, any of the systems or devices disclosed herein. For example, a user may be a healthcare provider or healthcare technician, or a parent or guardian assisting or monitoring a patient.

Various embodiments disclosed herein are directed to a device that monitors various patient parameters such as respiration rate, tidal volumes, pressure, CO₂ levels, and O₂ levels during a resuscitation process. Although the present disclosure discusses respiration rate, tidal volumes, pressures, CO₂ levels, and O₂ levels in particular, one of skill in the relevant art would recognize that other embodiments may include devices that monitor other or additional parameters during patient resuscitation as well. The device may be incorporated with a bag valve mask (BVM), a bag and endotracheal tube, or other resuscitation equipment, such as a mechanical ventilator. The device includes adjustable control settings that correspond to dimensions, e.g., height and weight, gender, and/or age of a given patient, allowing the device to warn a user if proper ventilation rates and tidal volumes are not being delivered. Some embodiments of the device are particularly advantageous for use in airway management in children, incorporating a Broselow Tape system into the adjustable control settings. The Broselow Tape is a color coded tape corresponding to established ranges of pediatric patient heights. Each color is associated with proper ventilation techniques and other important medical procedures specific to a given size range (e.g., an appropriate ventilation rate and tidal volume for a given range of pediatric patient heights). In addition, some embodiments include a sensor that measures end tidal CO₂. Measuring end tidal CO₂ allows a user to determine whether the patient has a pulse (e.g., after cardiac arrest), monitor cardiac output and ventilations, and determine whether an associated endotracheal tube is properly disposed in a trachea (i.e., as opposed to an esophagus). The device may also include a sensor that measures oxygen concentration and/or a sensor that measures temperature of the airflow. In operation, a user may identify a color group their patient corresponds to on the Broselow Tape, and may adjust the control settings on the device corresponding to that color group. A user may also identify the height and sex of a patient and the device will adjust the control settings for that height and sex. The user may then start ventilating the patient; if the ventilation is too fast or too slow, an alarm system will be triggered, alerting the user to look at an associated display to see how the ventilation should be adjusted.

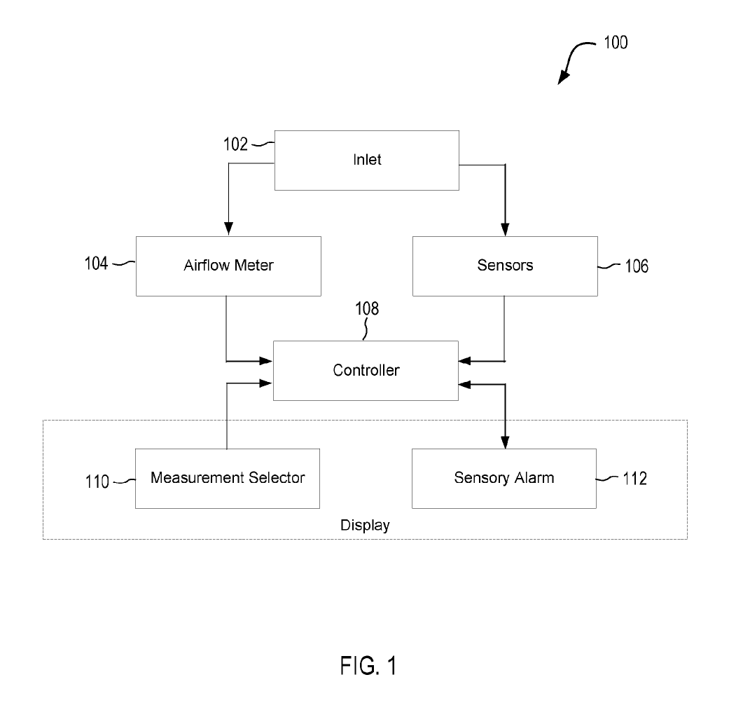

Referring now to FIG. 1, resuscitation and ventilation monitoring device 100 includes inlet 102, airflow meter 104, sensors 106, controller 108, measurement selector 110, and sensory alarm 112. In some arrangements, device 100 is incorporated into a BVM (e.g., disposed in line with airflows exchanged from a pump or bag) or some other resuscitation device, e.g., a bag and endotracheal tube or a mechanical ventilation device. Inlet 102 is configured to enable device 100 to be in fluid communication with the lungs of a patient. In some arrangements, inlet 102 is disposed in line with a conduit providing and receiving airflows with a patient mouth (e.g., disposed in line in a BVM). In some arrangements, inlet 102 is a mouthpiece configured to sealingly and removably engage the patient mouth. In some arrangements, inlet 102 is in fluid communication with airflows exchanged directly with a patient trachea. Consistent among these and other arrangements, inlet 102 enables airflows exchanged with the patient to pass through device 100.

Airflow meter 104 is configured to detect and measure respiration frequency and airflow volumes passing through inlet 102. As such, airflow meter 104 provides device 100 with data corresponding to a patient respiratory rate and tidal volumes. For example, the airflow meter 104 may be configured to measure respiratory rates ranging from 1-75 breaths per minute (“bpm”) and tidal volumes of 5-5,000 mL. In addition, airflow meter 104 may be configured to detect, measure, and compare the volume of an inhaled breath versus an exhaled breath of the patient within a breath cycle, such that the difference, i.e., A, may then be displayed on the user interface of device 100. Similarly, sensors 106 may include, for example, a CO₂ sensor configured to detect and measure CO₂ levels in airflows passing through inlet 102. For example, sensors 106 may be configured to measure CO₂ levels ranging from 0-99 mmHg. Sensors 106 may include a pressure sensor that measures the pressure within the airflow passing through the device. Sensors 106 may include an O₂ sensor configured to detect and measure O₂ levels in airflows passing through inlet 102. Additionally or alternatively, sensors 106 may include a temperature sensor configured to detect and measure the temperature of the airflow passing through inlet 102.

Measurement selector 110 is an input component that allows the user to provide device 100 with information relating to the size, gender, and/or age of the patient. In some arrangements, measurement selector 110 includes a plurality of preset buttons, toggles, switches, or other mechanically or digitally interactive components corresponding to pre-set increments of patient sizes (e.g., corresponding to colors of the Broselow Tape or direct measurement of the patient). In other arrangements, measurement selector 110 allows the user to manually enter specific patient size measurements, gender, and/or ages (e.g., a keyboard or a numerical pad, as disposed on a mechanical set of keys or a digital touchscreen), thereby allowing for a greater level of granularity.

Sensory alarm 112 is an output component configured to communicate with the user when at least one parameter (e.g., measured respiratory rate, tidal volume, pressure, difference in inhaled and exhaled volumes, CO₂ levels, or O₂ levels) is above or below acceptable levels (i.e., as determined by patient size, gender, and/or age information provided through measurement selector 110). In some arrangements, sensory alarm 112 is configured to provide the user with auditory signals (e.g., a beep, a tone, etc.). In some arrangements, sensory alarm 112 is configured to provide the user with visual signals (e.g., an illuminated light such as a lit LED or filament bub, or a message on a digital display, etc.).

Further, in some arrangements, sensory alarm 112 and/or measurement selector 112 may be incorporated into display 114. Display 114 is a digital screen configured to provide information to a user (e.g., an LCD screen). In some such arrangements, display 114 may include a touchscreen component disposed on device 100, allowing the user to both view measurement information (e.g., respiratory rates, tidal volumes, ventilation waveforms, pressure, difference in inhaled and exhaled volumes, temperatures, CO₂ levels, O₂ levels) and acceptable ranges of such measurements, as well as provide device 100 with measurement information (e.g., patient sizes, patient gender, patient ages, Broselow Tape color selections, etc.). The measurement information may include color coded alarms to indicate if the ventilation is on-target or off-target, and percentages of breaths that are off-target. Sensory alarm 112 and/or display 114 may be part of a separate device remote from device 100, such that device 100 sends information to the sensory alarm of the separate remote device when at least one parameter is above or below an acceptable level to communicate with the user via auditory or visual signals. The remote device may further include a user interface having, for example, a measurement selector as described above for inputting patient information. Communication between device 100 and the user interface of the separate remote device may occur across multiple platforms, e.g., WiFi, Bluetooth, Wixel-based communication, and cellular communication. In addition, the communication between device 100 and the user interface of the separate remote device may occur across a variety of ranges, e.g., short (feet) or distant (miles).

Controller 108 includes data processing and non-transient storage hardware and associated logics to perform various functions described herein. Data processing hardware, e.g., a processor, may include and/or be coupled to non-transient storage hardware having instructions, e.g., algorithms, stored thereon that when executed by the processor cause the processor, and thereby controller 108, to perform various functions. For example, controller 108 may be configured to receive a user input from measurement selector 110 corresponding to a patient size (e.g., a Broselow Tape color, or numerical measurements of height and weight), patient gender, and/or patient age. Controller 108 may then determine or retrieve acceptable measurement ranges for the patient size, gender, and/or age (e.g., as stored in a non-transient storage medium such as a flash drive, or as determined by a measurement calculation logic). Airflows from the patient may then pass through inlet 102, causing airflow meter 104 and sensors 106 to provide controller 108 with airflow measurements. The data processing hardware of controller 108 may calculate or process corresponding respiratory rates, tidal volumes, pressures, differences in inhaled and exhaled volumes, CO₂ and O₂ levels, and temperatures upon execution of the instructions stored thereon, compare those values with the acceptable measurement ranges, and cause sensory alarm 112 to alert the user if the airflow measurements fall outside the acceptable measurement ranges. The processed information may be in the form of pressure and/or flow waveforms, such that the waveforms are displayed on the user interface for observation. Controller 108 may store airflow measurement information and/or calculated or processed information in the non-transitory storage medium, such that the stored information may be downloaded at a later time for analysis.

Device 100 may be configured to work within a network of devices and user interfaces. For example, device 100 may be placed in line to measure flow, and process and send information to remote locations where a remote user device having a user interface, e.g., a tablet, phone, computer, or a “heads up display” such as Google glasses, communicates the information to the user. In some such arrangements, multiple devices may send information to a single user interface. Accordingly, calculations may be executed by controller 108 on device 100 itself, or raw data streams from airflow meter 104 and sensors 106 may be sent to a remote controller of a separate device where calculations are executed and displayed to the user. Communication between device 100 and the user interface of the separate remote device may occur across multiple platforms, e.g., WiFi, Bluetooth, Wixel-based communication, and cellular communication. In addition, the communication between device 100 and the user interface of the separate remote device may occur across a variety of ranges, e.g., short (feet) or distant (miles). Components of the device and several arrangements thereof are discussed in more detail below.

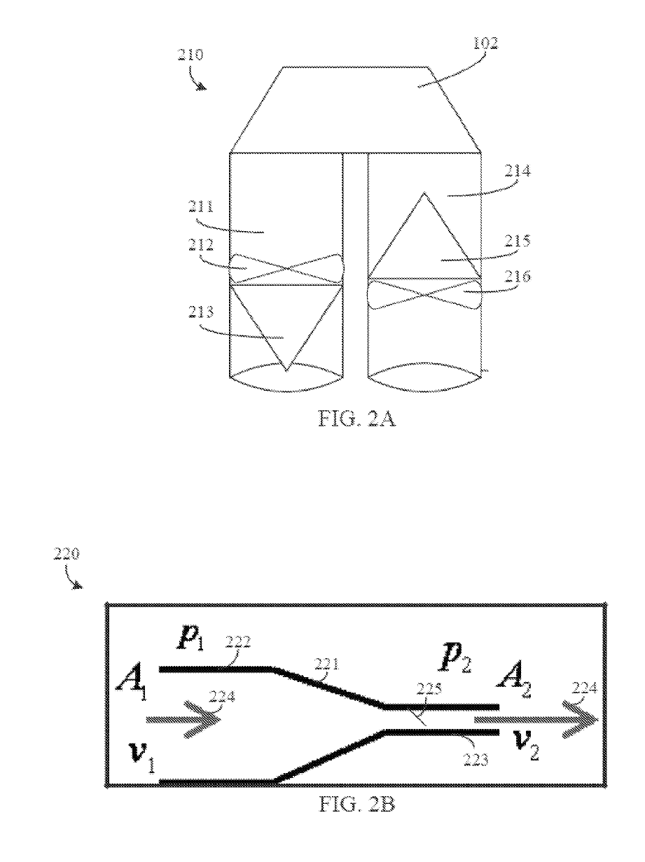

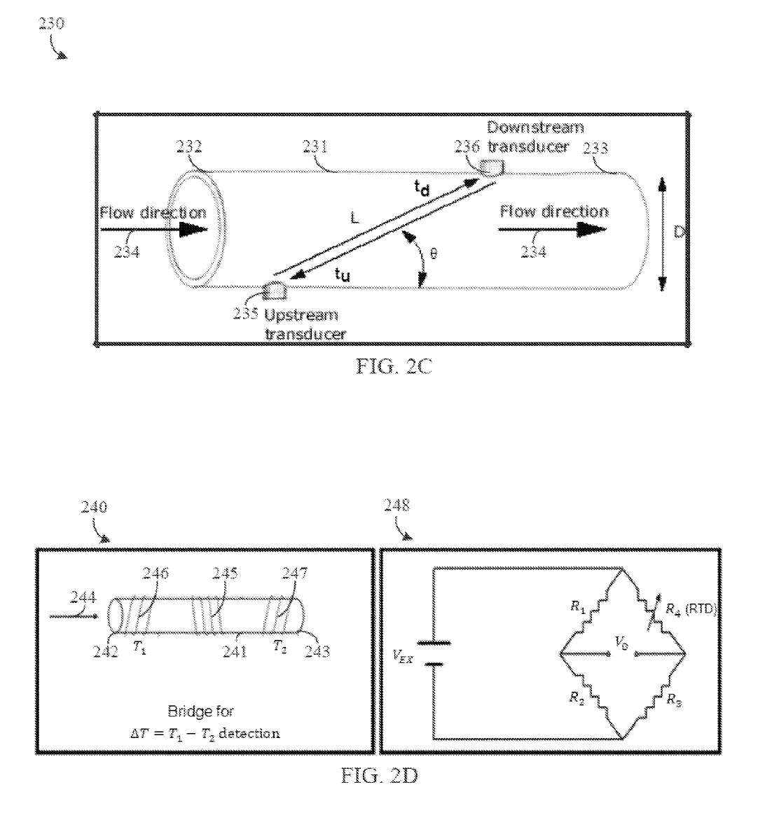

Referring now to FIG. 2A, first airflow meter arrangement 210 incorporates the use of fans, and includes outflow chamber 211 and inflow chamber 214. Outflow chamber 211 includes one-way outflow valve 213 and outflow fan 212. In turn, inflow chamber 214 includes a corresponding one-way inflow valve 215 and inflow fan 216. Each of outflow chamber 211 and inflow chamber 214 are in fluid communication with inlet 102, which is in line with airflows exchanged with the lungs of a patient.

In first airflow meter arrangement 210, air pumped into the patient's lungs flows through inflow chamber 214, and air withdrawn out of the patient's lungs flows through outflow chamber 211, as a result of each respective one-way valve. As air flows through a given chamber (e.g., outflow chamber 211), the associated fan (e.g., outflow fan 212) will spin. Each fan includes a magnet attached to a fan axle, generating a current while the fan spins. The generated current may pass over a fan resistor where a voltage may be measured. As such, a time between output voltage peaks may be used to determine a ventilation rate. The area underneath a voltage curve of inflow fan 216 corresponds to the volume of air delivered to the lungs of the patient, and the area underneath a voltage curve of outflow fan 212 corresponds to the volume of air withdrawn from the lungs of the patient. The difference between the volumes of air delivered and withdrawn (i.e., passing through inflow chamber 214 and outflow chamber 211, respectively) may indicate the presence and extent of any air leaks (e.g., in the device 100 itself, at a ventilation mask, at an endotracheal tube, etc.).

Outflow fan 212 and inflow fan 216 should be comprised of materials that may survive temperature and moisture conditions present during patient resuscitation. Acceptable fan materials include, for example: glass reinforced polypropylene (PPG); glass reinforced polyamide (PAG—Nylon); glass reinforced polyamide, industrial quality (PAGI); electro anti-static glass reinforced (PAGAS - Nylon); vibration stabilized glass reinforced polyamide (PAGST—“Super Tuff” Nylon); and aluminum, EN AC-AL SI12CU1 (FE) (AL).

Referring now to FIG. 2B, second airflow meter arrangement 220 incorporates the use of a volumetric flowmeter. Second airflow meter arrangement 220 includes airflow conduit 221 with upstream end 222 having a first area and downstream end 223 having a smaller second area (i.e., relative to the first area). Airflow 224 (e.g., traveling to or from the lungs of a patient) travels from upstream end 222 to downstream end 223. Given the difference in area, airflow 224 exhibits a lower first velocity and a lower first pressure at upstream end 222, and a corresponding higher second velocity and a higher second pressure at downstream end 223. In one arrangement, articulating pressure flap 225 is disposed perpendicularly to the direction of airflow 224 in downstream end 223. Pressure flap 225 is configured to pivot across a range of motion corresponding to an airflow pressure exerted upon it, thereby measuring the pressure at downstream end 223. As such, in second airflow meter arrangement 220, the respiration rate may be determined from the oscillation of pressure flap 225, and the tidal volume may be determined from the known values (e.g., the first and second volume) and measured values (e.g., the first and second pressure and the first and second velocity) as applied to Bernoulli's equation:

1/2ρv²+ρgz+p=constantv₂²=v₁²·(ρ1A1/ρ2A2)²

where ρ, v, and ρ represent density, velocity and pressure of the airflow, respectively.

Referring now to FIG. 2C, third airflow meter arrangement 230 incorporates ultrasonic transducers to measure tidal volumes and respiratory rates. Third airflow meter arrangement 230 includes airflow conduit 231 having upstream end 232 and downstream end 233. Airflow 234 (e.g., traveling to or from the lungs of a patient) travels from upstream end 232 to downstream end 233. First ultrasonic transducer 235 is disposed in airflow conduit 231 towards upstream end 232, and second ultrasonic transducer 236 is disposed in airflow conduit 231 towards downstream end 233 (i.e., relative to first ultrasonic transducer 235). Each transducer emits and receives sound in alternating directions. When airflow 234 is present in airflow conduit 231, the time it takes for acoustic waves to travel from first ultrasonic transducer 235 to second ultrasonic transducer 236, t_{d} (i.e., acoustic waves traveling with airflow 234), is shorter than from second ultrasonic transducer 236 to first ultrasonic transducer 235, tᵤ (i.e., acoustic waves traveling against airflow 234). This difference in time, Δt, is proportional to the velocity of airflow 234, and airflow volume may also be calculated in the following manner:

### Mathematical Model

V=L²Δt/2Xtᵤt_{d}
V: Flow velocity
L: Distance between the transducers
X: Projected length of the path along the value (X=L cos Θ)
tᵤ: time for wave signal to travel upstream
t_{d}: time for the wave signal to travel downstream

### Volumetric Flow

Q=VA

The transit time of each sound pulse from each transducer may be precisely measured with a digital clock.

In third airflow meter arrangement 230, airflow conduit 231 may be disposable since it may be configured to have no sensor elements exposed to airflow 234 and/or to have no moving parts. In such an arrangement, airflow conduit 231 acts only as a hygienic shield and is transparent to the ultrasonic pulses traveling between the transducers. Potential advantages of third airflow meter arrangement 230 include sensor elements that are not directly in contact with gas flow, and measurement data that is relatively insensitive to other factors such as temperature, pressure, density and viscosity of fluids.

Referring now to FIG. 2D, fourth airflow meter arrangement 240 incorporates mass airflow sensors. Fourth airflow meter arrangement 240 includes airflow conduit 241 having upstream end 242 and downstream end 243. Airflow 244 (e.g., traveling to or from the lungs of a patient) travels from upstream end 242 to downstream end 233. In addition, heater circuit 245 is disposed between upstream temperature sensor 246 and downstream temperature sensor 247, each of which are annularly disposed about airflow conduit 241.

In operation, a predetermined amount of heat is applied to heater circuit 245. Upstream temperature sensor 246 and downstream temperature sensor 247 are each not directly heated, and as such, act as reference points to heater circuit 245. When there is no flow through airflow conduit 241, the differences in temperatures between heater circuit 245 and each of upstream temperature sensor 246 and downstream temperature sensor 247 are at their greatest. As airflow 244 flows through airflow conduit 241, heater circuit 245 cools and the differences in temperatures between heater circuit 245 and each of upstream temperature sensor 246 and downstream temperature sensor 247 decreases. In addition, as upstream temperature sensor 246 and downstream temperature sensor 247 are disposed on either side of heater circuit 245, resulting temperature differentials may indicate the direction of airflow 244 as well. Alternating directions of airflow 244 may thus be detected and give rise to respiration rates.

In some arrangements, dual Wheatstone bridge system 248 is disposed on airflow conduit 241 and incorporates heater circuit 241, upstream temperature sensor 246, and downstream temperature sensor 247 as a resistance-temperature detector (RTD). In an RTD, one of the resistance values will be dependent on the measured temperature differential. The output of the RTD is relatively linear with temperature, giving rise to a ratiometric output voltage that directly corresponds to the differential voltage across the Wheatstone bridge that is proportional to the mass flow.

Although four examples of airflow meter 104 have been provided in FIGS. 2A-2D, one of skill in the relevant art would recognize that other arrangements are possible. For example, airflow meter 104 may be implemented using IR spectrometry or ultrasound technology.

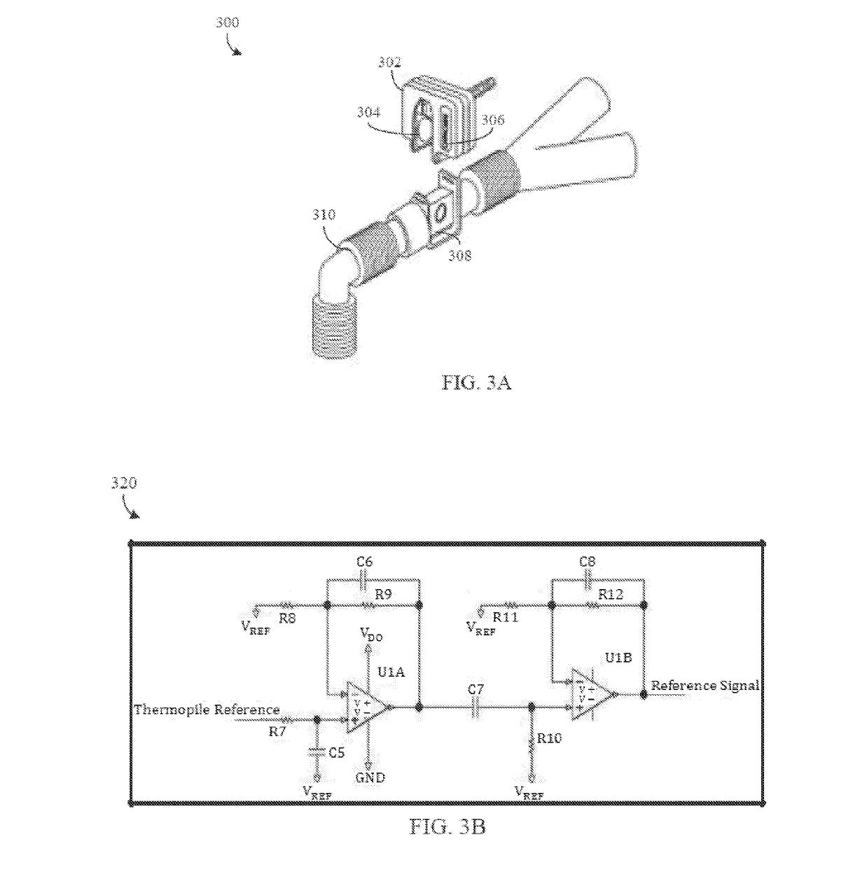

Referring now to FIG. 3A, example sensor arrangement 300 corresponding to sensors 106 incorporates IR spectrometry. Sensor arrangement 300 includes sensor housing 302, which serves as a foundation upon which sensor components are attached. IR source 304 is disposed in sensor housing 302 opposite IR detector 306. IR source 304 provides infrared light across an airflow exchanged with the lungs of a patient and to IR detector 306. In some arrangements, IR source 304 includes an IR filter configured to narrow the range of wavelengths passing through the airflow. Further, in some arrangements, a band-pass filter may be disposed within sensor housing 302 to remove all other wavelengths outside the absorption range of CO₂ or O₂ depending on the type of sensor (e.g., circuitry component 320 as shown in FIG. 3B). IR detector 306 may include a thermopile with a built-in filter correspondingly configured to detect IR intensity after passing through the airflow, and may thus determine the amount of CO₂ and O₂ in the airflow. Sensor housing 302 may be configured to engage corresponding adapter slot 308 disposed in line with airflow conduit 310. Adapter slot 308 is configured to allow IR source 304 to transmit infrared light across an airflow within airflow conduit 310 and to IR detector 306. In some arrangements, airflow conduit 310 includes one or more filters configured to remove water from the airflow.

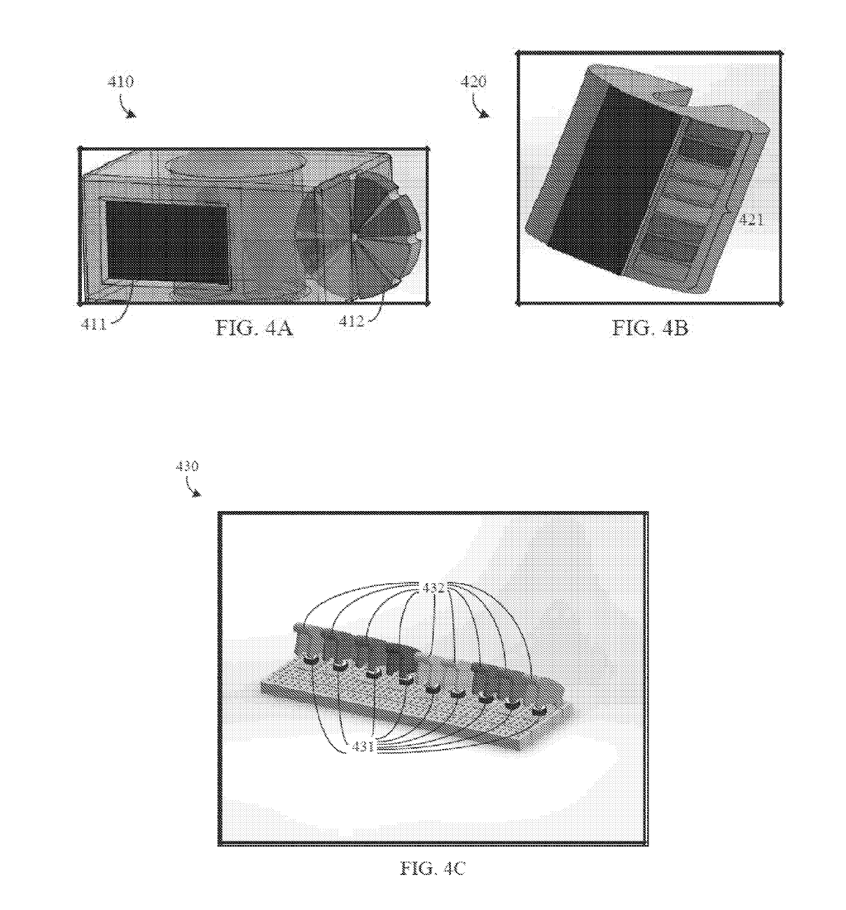

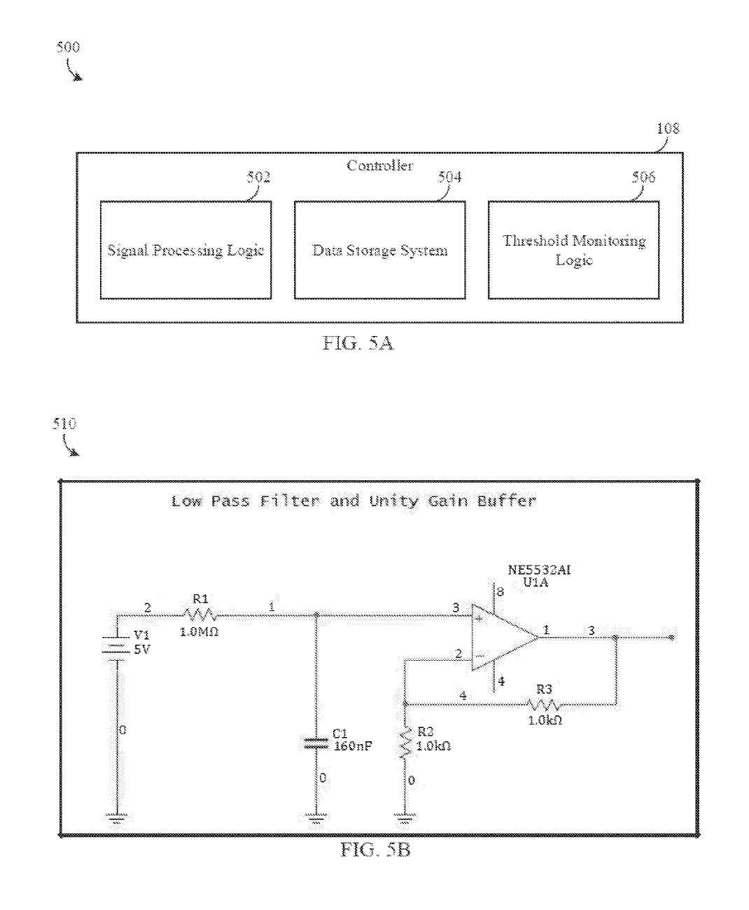

Referring now to FIG. 4A, first arrangement 410 of measurement selector 110 is shown. First arrangement 410 includes display 411 (e.g., display 114). Display 411 is a digital screen configured to provide a user with information relating to the operation of resuscitation and ventilation monitoring device 100 (e.g., measurement information, acceptable measurement ranges, etc.). In some arrangements, display 411 includes an input aspect such as a touchscreen or an associated keypad or keyboard. As such, in some such arrangements, the user may be able to manually enter precise patient measurements (e.g., a specific height and weight), gender, and/or age using display 411. Device 100 may be configured to use the manually entered patient measurements to categorize the patient in an appropriate group (e.g., one of the Broselow Tape colors, corresponding to a height and weight range that includes the specific height and weight entered), or to generate acceptable measurement ranges tailored to the patient's specific height and weight, gender, and/or age.

First arrangement 410 also includes dial 412 with selectable colors corresponding to the Broselow Tape. The Broselow Tape assigns different colors according to the size (e.g., height and weight) of a patient, which may be represented corresponding notched sections on dial 412. When the user selects a color using dial 412, device 100 will tell the user the appropriate ventilation rate and will alarm the user when ventilation is inadequate (e.g., via display 411). Pediatricians and other medical personnel may already be familiar with how the Broselow Tape is used, and as such, using dial 412 may be faster and easier than using display 411 to manually enter the height, sex, and weight values of a given patient. In addition to sections corresponding to colors of the Broselow Tape, dial 412 may include one or more notched sections that correspond to one or more adult sizes.

Referring now to FIG. 4B, as shown in second arrangement 420 of measurement selector 110, Broselow Tape settings may be assigned to some or all of plurality of pushbuttons 421 (i.e., instead of dial 412 of FIG. 4A). In addition, plurality of pushbuttons 421 may also include labels of heights corresponding to the Broselow Tape colors so a user may quickly select a correct setting during resuscitation. Further, as shown in third arrangement 430 of measurement selector 110, plurality of pushbuttons 431 may be protected from inadvertent actuation by corresponding plurality of switch covers 432.

Referring now to FIG. 5A, controller 108 includes signal processing logic 502, data storage system 504, and threshold monitoring logic 506. Signal processing logic 502 is configured to receive measurement data from airflow meter 104 and sensors 106. In one aspect, signal processing logic 502 is configured to receive measured CO₂ levels from sensors 106, and route the measured CO₂ levels to threshold monitoring logic 506. In another aspect, signal processing logic 502 is configured to receive measured O₂ levels from sensors 106, and route the measured O₂ levels to threshold monitoring logic 506. In yet another aspect, signal processing logic 502 is configured to receive measured temperatures from sensors 106, and route the measured temperatures to threshold monitoring logic 506. In yet another aspect, signal processing logic 502 is configured to receive measured airflow data from airflow meter 104, and route the airflow data to threshold monitoring logic 506. In yet another aspect, signal processing logic 502 is configured to receive measured pressure from sensors 106, and route the pressure data to threshold monitoring logic 506. In some arrangements, signal processing logic 502 is further configured to calculate respiration rates, tidal volumes, and the difference in inhaled and exhaled volume, e.g., A, from the measured airflow data (e.g., as discussed above with respect to FIGS. 2A-2D), and forward the respiration rates, tidal volumes, and A to threshold monitoring logic 506.

In some arrangements, the output voltage generated by sensors at airflow meter 104 and sensors 106 may be in the range of about 5Vdc±0.36 Vdc at 200 SLPM (standard liters per minute), and as such, no signal amplification is required. The frequency range corresponding to human respiratory rate may be in the range of about 0.01 to 1.0 Hz. Hence, a low pass filter followed by a unity gain voltage buffer with the specifications (e.g., as shown by low pass filter circuit 510 in FIG. 5B) may be used as part of signal processing logic 502 to eliminate the noise and adjust the output impedance. Further, in some arrangements, the input signal from the sensors is analog and signal processing logic 502 may also be configured to perform an analog to digital conversion (e.g., 8/16-channel, 10-bit ADC).

Data storage system 504 is an on-board storage medium configured to retrievably maintain data, for example, data corresponding to ranges of acceptable CO₂ levels, O₂ levels, tidal volumes, respiration rates, pressures, and differences in inhaled and exhaled volume for a plurality of patient sizes. In some arrangements, the ranges are organized by categories corresponding to colors of the Broselow Tape. In some arrangements, acceptable ranges for adults are stored on data storage system 504 as well. Further, in some arrangements, data storage system 504 may include calculation algorithms for determining specific ranges for CO₂ levels, O₂ levels, tidal volumes, respiration rates, and differences in inhaled and exhaled volume for specific patient heights and weights, gender, and/or age. Data storage system 504 may store the data so that the stored data may be downloaded at a later time for analysis.

Threshold monitoring logic 506 allows controller 108 to interface with a user of device 100. For example, threshold monitoring logic 506 may be configured to receive a user input from measurement selector 110 corresponding to a patient's height and weight (e.g., a Broselow Tape color, or a specific height and weight), gender, and/or age. Threshold monitoring logic 506 may then retrieve appropriate respiratory rate, tidal volume, difference in inhaled and exhaled volume, CO₂ level, and O₂ level ranges from data storage system 504. Where a specific patient height and weight, gender, and/or age is provided in the user input, threshold monitory logic 506 may retrieve and execute a calculation algorithm from data storage system 504 to determine appropriate ranges. In some arrangements, threshold monitoring logic 506 causes a display (e.g., display 114) to present the user input and the ranges to the user.

Threshold monitoring logic 506 receives measurement data (e.g., respiratory rates, tidal volumes, pressures, differences in inhaled and exhaled volume, CO₂ levels, and O₂ levels) from signal processing logic 502 and compares the measurement data with the respiratory rate, tidal volume, difference in inhaled and exhaled volume, CO₂ level, and O₂ level ranges appropriate for the patient's size, gender, and/or age. In some arrangements, if at least one these measurement data types falls above or below a respective range, threshold monitoring logic 506 causes sensory alarm 112 to notify the user that ventilation currently being applied is not appropriate for the patient's size, gender, and/or age. In some such arrangements, the threshold monitoring logic causes display 114 to provide the user with information relating to current measurement data and whether the current measurement data falls above or below an appropriate range.

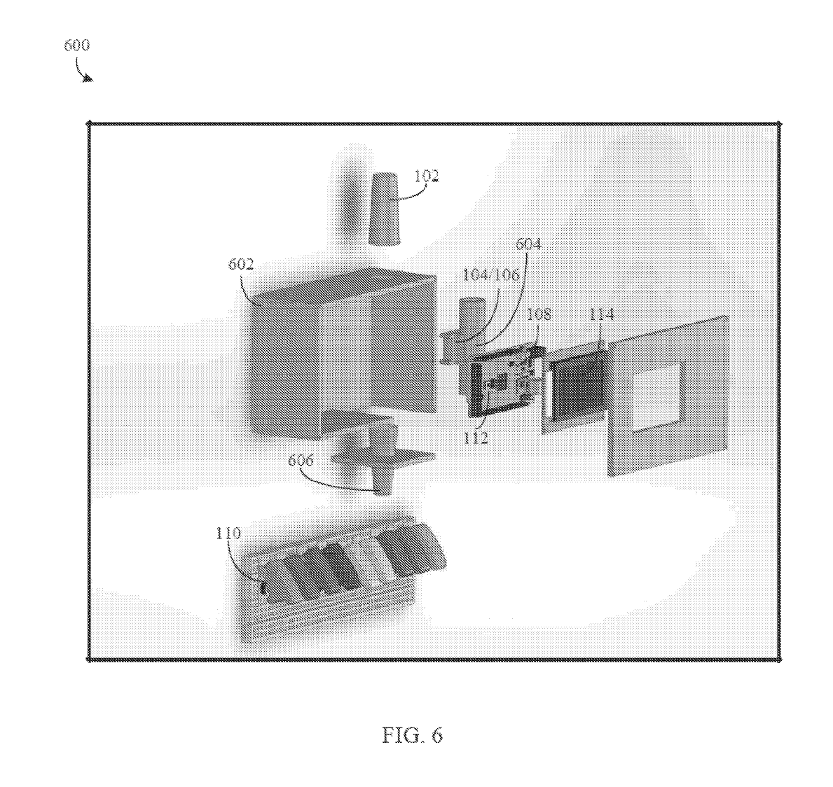

Referring now to FIG. 6, example embodiment 600 of device 100 is shown. In example embodiment 600, inlet 102 is communicatively engaged to airflow conduit 604, which in turn is communicatively engaged to outlet 606. Inlet 102 may be further engaged to a mouthpiece or other adapter configured to removably engage a patient airway. Outlet 606 may be engaged to a pressure manipulation device, for example a BVM or a mechanical ventilator. Airflow conduit 604 houses airflow meter 104 and sensors 106, and bridges inlet 102 to outlet 606.

In example embodiment 600, inlet 102 and outlet 606 are disposed on the exterior of enclosure 602, while airflow conduit 604 is disposed in the interior of enclosure 602. Enclosure 602 is a protective housing and foundation for various components of device 100. Enclosure 602 may be made up of any several types of materials (e.g., plastic, acrylic, metal, or alloys thereof) and may be assembled in various ways (e.g., snapped together at a plurality of pegs and slots, fastened via bolts or screws, glued, etc.).

Controller 108 is disposed within enclosure 602. Controller 108 may be embodied as, for example, an Arduino Mega 2560 8-bit microcontroller or other suitable programmable microcontroller. In addition to data processing hardware, the Arduino Mega 2560 includes 128 KB of flash memory (i.e., data storage system 504). Further, in example embodiment 600, controller 108 includes sensory alarm 112 mounted on an associated circuit board, for example as a flashing LED and/or a speaker.

Example embodiment 600 further includes display 114 embodied as a digital (e.g., LCD) screen. Display 114 is electrically engaged to controller 108, and as such may be configured to provide a user with measurement, range, waveforms, and alert information.

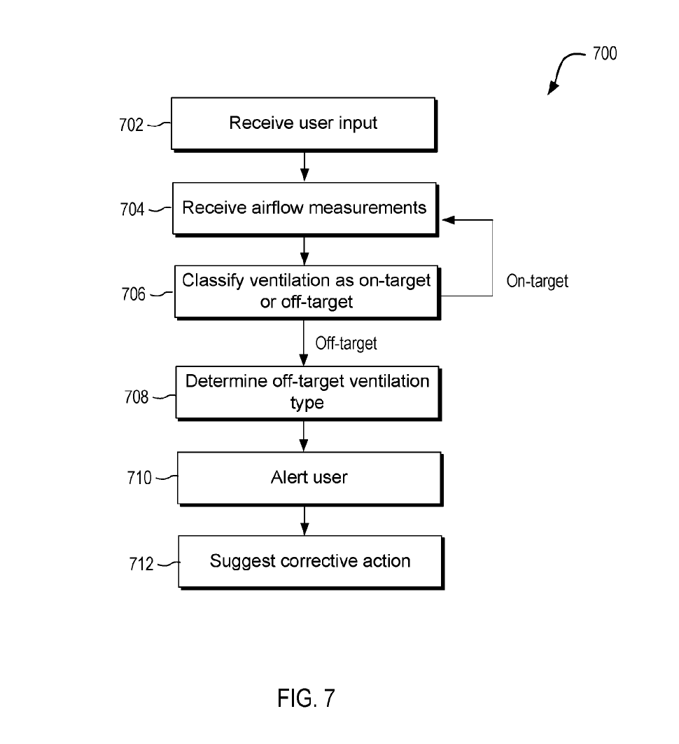

Referring now to FIG. 7, flow chart 700 illustrates the actions performed by controller 108 of device 100 coupled to a ventilator. The controller identifies ideal ventilation conditions using preset definitions followed by comparisons to current ventilation measurements to provide clinical decision support. Initially, at step 702, controller 108 receives user input from a user via measurement selector 110. Next, at step 704, controller 108 receives airflow measurements from airflow meter 104 and/or one or more sensors 106 as described above. At step 706, controller 108 classifies the ventilation as on-target or off-target. To determine whether the ventilation is on-target, e.g., whether the ventilation observed is within normal limits for the patient given the size and weight measurements, gender, and/or age of the patient inputted in measurement selector 110, controller 108 identifies the tidal volumes as described above. Using patient size and weight measurements, gender, and/or age, controller 108 may determine the level of appropriateness by comparing the measured tidal volumes and the ideal tidal volumes. As a result, controller 108 determines that the observed ventilation is on-target if the level of appropriateness is within normal limits for the patient given the size and weight measurements, gender, and/or age of the patient. If controller 108 determines that the observed ventilation is on-target at step 706, controller 108 returns to step 704 and device 100 continues to resuscitate and monitor the patient coupled to the mechanical ventilator. If controller 108 determines that the observed ventilation is off-target, e.g., the level of appropriateness is not within normal limits for the patient given the size and weight measurements, gender, and/or age of the patient, controller 108 proceeds to step 708.

At step 708, controller 108 determines the type of off-target ventilation observed. Types of off-target ventilation include, but are not limited to, tidal volume violations, pressure violations, and work of breathing violations. At step 710, controller 108 and sends information to sensor alarm 112 to communicate an alert to the user. Depending on the type of off-target ventilation observed, controller 108 directs sensor alarm 112 to communicate specific alerts to the user. Finally, at step 712 controller 108 may suggest corrective actions for the user via the user interface of device 100 to execute the adjustments required to bring the observed ventilation within normal limits for the patient given the size and weight measurements, gender, and/or age of the patient.

Upon receiving the corrective action recommendations from device 100, the user may adjust the ventilator, e.g., manual bagging of the patient or the ventilator settings of a mechanical ventilator, to bring the observed ventilation within normal limits for the patient given the size and weight measurements, gender, and/or age of the patient.

Those of skill in the art will appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

In one or more example embodiments, the functions described may be implemented in hardware, software, or firmware executed on a processor, or any combination thereof. For example, certain embodiments may comprise a computer program product for performing the operations presented herein. Such a computer program product may comprise a computer-readable medium having instructions stored and/or encoded thereon, the instructions being executable by one or more processors to perform the operations described herein. When the functions described herein are implemented in software, the functions may be stored on or transmitted over as one or more instructions or code on a computer-readable medium. Computer readable media includes both computer storage media and communication media including any medium that facilitates transfer of a computer program from one place to another. A storage media may be any available media that may be accessed by a computer. By way of example, and not limitation, such computer-readable media may comprise RAM, ROM, EEPROM, CD-ROM or other optical disk storage, magnetic disk storage or other magnetic storage devices, or any other medium that may be used to carry or store desired program code in the form of instructions or data structures and that may be accessed by a computer. Also, any connection is properly termed a computer-readable medium. For example, if the software is transmitted from a web site, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, includes compact disc (CD), laser disc, optical disc, digital versatile disc (DVD), and Blu-ray disc where disks usually reproduce data magnetically, while discs reproduce data optically with lasers. Combinations of the above should also be included within the scope of computer-readable media.

Further, it should be appreciated that modules and/or other appropriate means for performing the methods and techniques described herein may be downloaded and/or otherwise obtained by a device as applicable. For example, such a device may be coupled to a server to facilitate the transfer of means for performing the methods described herein. Alternatively, various methods described herein may be provided via storage means (e.g., RAM, ROM, a physical storage medium such as a compact disc (CD) or flash drive, etc.), such that a device may obtain the various methods upon coupling or providing the storage means to the device. Moreover, any other suitable technique for providing the methods and techniques described herein to a device may be utilized.

Although the foregoing has included detailed descriptions of some embodiments by way of illustration and example, it will be readily apparent to those of ordinary skill in the art in light of the teachings of these embodiments that numerous changes and modifications may be made without departing from the spirit or scope of the appended claims.

What is claimed:

1. A resuscitation and ventilation monitoring system, the system comprising:
an inlet in fluid communication with airflows exchanged with lungs of a patient;
an airflow meter in fluid communication with and collecting airflow measurements from the inlet;
a sensory alarm selectively providing an alert to a user of the system;
a measurement selector including selectable increments of patient height; and
a controller communicatively engaged to each of the airflow meter, the sensory alarm, and the measurement selector,
wherein the controller receives a user input from the measurement selector and determines a corresponding range of acceptable respiratory rates and tidal volumes;
wherein the controller determines a patient respiratory rate and tidal volumes from the airflow measurements received from the airflow meter; and
wherein the controller causes the sensory alarm to provide the alert to the user when at least one of the patient respiratory rate or the tidal volumes is outside the corresponding range of acceptable respiratory rates and tidal volumes.

2. The resuscitation and ventilation monitoring system of claim 1, further comprising:
a CO₂ sensor in fluid communication with and collecting CO₂ level measurements from the inlet, wherein the controller is communicatively engaged to and receives CO₂ level measurements from the CO₂ sensor; and
a display, wherein the controller provides the user with the CO₂ level measurements via the display.

3. The resuscitation and ventilation monitoring system of claim 2,
wherein the controller determines a range of acceptable CO₂ levels; and
wherein the controller causes the sensory alarm to provide the alert to the user when the CO₂ level measurements are outside the corresponding range of acceptable CO₂ levels.

4. The resuscitation and ventilation monitoring system of claim 1, further comprising:
an O₂ sensor in fluid communication with and collecting O₂ level measurements from the inlet, wherein the controller is communicatively engaged to and receives 0₂ level measurements from the O₂ sensor; and
a display, wherein the controller provides the user with the O₂ level measurements via the display.

5. The resuscitation and ventilation monitoring system of claim 1, further comprising:
a pressure sensor in fluid communication with and collecting pressure measurements from the inlet, wherein the controller is communicatively engaged to and receives pressure measurements from the pressure sensor; and
a display, wherein the controller provides the user with the pressure measurements via the display.

6. The resuscitation and ventilation monitoring system of claim 5,
wherein the controller determines the range of acceptable pressures; and
wherein the controller causes the sensory alarm to provide an alert to the user when the pressure measurements are outside the corresponding range of acceptable pressure levels.

7. The resuscitation and ventilation monitoring system of claim 1, wherein the sensory alarm provides a visual alert to the user.

8. The resuscitation and ventilation monitoring system of claim 1, wherein the sensory alarm provides an audible alert to the user.

9. The resuscitation and ventilation monitoring system of claim 1,
wherein the measurement selector includes a plurality of colored options, each of the plurality colored options corresponding to colors and associated measurement increments defined by the Broselow Tape; and
wherein each of the colors and associated measurement increments defined by the Broselow Tape correspond to a respective range of acceptable respiratory rates and tidal volumes.

10. The resuscitation and ventilation monitoring system of claim 9, wherein the measurement selector includes a plurality of switches, wherein each of the plurality switches corresponds to one of the plurality of colored options.

11. The resuscitation and ventilation monitoring system of claim 9, wherein the measurement selector includes a rotatable dial divided into a plurality of segments, wherein each of the plurality of segments corresponds to one of the plurality of colored options.

12. The resuscitation and ventilation monitoring system of claim 9, wherein the measurement selector includes a digital screen configured to display colored digital representations, wherein each colored digital representation corresponds to one of the plurality of colored options.

13. The resuscitation and ventilation monitoring system of claim 9,
wherein the measurement selector further includes at least one colored option corresponding to measurement increments for an adult patient; and
wherein each of the at least one colored option corresponding to measurement increments for an adult patient are associated with a respective range of acceptable respiratory rates and tidal volumes.

14. The resuscitation and ventilation monitoring system of claim 1, wherein the measurement selector further includes selectable increments of at least one of patient weight, patient gender, or patient age.

15. The resuscitation and ventilation monitoring system of claim 1, wherein the controller is coupled to a remote device.

16. The resuscitation and ventilation monitoring system of claim 15, wherein the airflow meter communicates the airflow measurements to the controller via at least one of WiFi, Bluetooth, Wixel-based communication, or cellular communication.

17. The resuscitation and ventilation monitoring system of claim 1, wherein the controller is further configured to compare an amount of breath inhaled with an amount of breath exhaled from the airflow measurements received from the airflow meter, and wherein the controller causes the sensory alarm to provide the alert to the user when the compared amount of breath inhaled and exhaled is outside a predetermined threshold.

18. The resuscitation and ventilation monitoring system of claim 1, wherein the controller further comprises a data storage configured to store data comprising at least one of the corresponding range of acceptable respiratory rates, the corresponding range of acceptable tidal volumes, the patient respiratory rate, or the patient tidal volumes, wherein the data is configured to be downloaded and analyzed at a later time.

19. The resuscitation and ventilation monitoring system of claim 1, further comprising a display,
wherein the display provides the user with at least one of a selected increment of patient height, a selected Broselow Tape color, the patient respiratory rate, the patient CO₂ level, the patient O₂ level, a calculated difference in inhaled and exhaled volume, or the patient total tidal volumes.

20. The resuscitation and ventilation monitoring system of claim 19, wherein the display provides the user with at least one of the patient respiratory rate or the patient tidal volumes in the form of ventilation waveforms.

21. The resuscitation and ventilation monitoring system of claim 19, wherein the sensory alarm provides a visual alert to the user through the display.

22. The resuscitation and ventilation monitoring system of claim 19,
wherein the display is a touchscreen; and
wherein the measurement selector is disposed in the display.

23. A non-transitory computer-readable medium having instructions that, when executed by a processor of a ventilator, cause the processor to:
receive user input from a user, the user input comprising at least one of patient height, weight, gender, or age;
receive airflow measurements indicative of ventilation of a patient from at least one of an airflow meter or one or more sensors;
classify ventilation as either on-target or off-target based on whether the ventilation is within a predetermined limit defined by the user input;
generate an alert if the ventilation is off-target; and
suggest corrective action via a user interface if the alert is generated.

24. The non-transitory computer-readable medium of claim 20, wherein the instructions, when executed by a processor, cause the processor to suggest corrective action that may be implemented by the user to adjust a manual bagging of the patient or ventilator settings of a mechanical ventilator.
